(19) United States
(12) Patent Application Publication
(10) Pub. No.: US 2024/0413397 A1
(43) Pub. Date: Dec. 12, 2024

(54) ELECTROLYTE FOR RECHARGEABLE LITHIUM BATTERY AND RECHARGEABLE LITHIUM BATTERY INCLUDING THE SAME

(71) Applicant: SAMSUNG SDI CO., LTD., Yongin-si (KR)

(72) Inventors: Tae Jin LEE, Yongin-si (KR); Si-Young CHA, Yongin-si (KR); Inah KANG, Yongin-si (KR); Minji SIM, Yongin-si (KR); Hanmam JEONG, Yongin-si (KR)

(21) Appl. No.: 18/646,626

(22) Filed: Apr. 25, 2024

(30) Foreign Application Priority Data
Jun. 8, 2023 (KR) 10-2023-0073678

(51) Int. Cl. H01M 10/0567; C07D 405/14; C07F 9/06; C07F 9/6574; H01M 10/0525; H01M 10/0569

(52) U.S. Cl. CPC H01M 10/0567; C07D 405/14; C07F 9/062; C07F 9/65742; H01M 10/0525; H01M 10/0569; H01M 2300/004

(57) ABSTRACT

An electrolyte for a rechargeable lithium battery and a rechargeable lithium battery including the same are provided. The electrolyte includes a non-aqueous organic solvent; a lithium salt; a first additive represented by Chemical Formula 1, and a second additive represented by Chemical Formula 2.

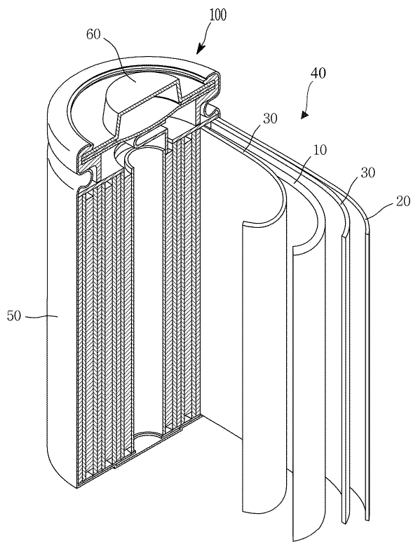

### CROSS-REFERENCE TO RELATED APPLICATION

This application claims priority to and the benefit of Korean Patent Application No. 10-2023-0073678, filed on Jun. 8, 2023, in the Korean Intellectual Property Office, the entire content of which is incorporated herein by reference.

### BACKGROUND

### 1. Field

According to one or more embodiments, the present disclosure relates to an electrolyte for a rechargeable lithium battery and a rechargeable lithium battery including the same.

### 2. Description of the Related Art

A rechargeable lithium battery may be recharged and has three or more times the (higher)energy density per unit weight as compared to a comparable lead storage battery, nickel-cadmium battery, nickel hydrogen battery, nickel zinc battery and/or the like. The rechargeable lithium battery may be also charged at a relatively high rate and thus, is commercially manufactured for a laptop, a cell phone, an electric tool, an electric bike, and/or the like, and researches on improvement of additional energy density have been actively made or pursued.

Such a rechargeable lithium battery is manufactured by injecting an electrolyte into an electrode assembly, which includes a positive electrode (e.g., including a positive electrode active material) and a negative electrode (e.g., including a negative electrode active material).

Recently, one developmental advance in the technology of rechargeable lithium batteries is to improve relatively high-temperature performance characteristics. In general, the rechargeable lithium batteries may experience an increase in electrical resistance at a relatively high temperature, that may increase the likelihood of an ignition and/or explosion. For example, in a module and/or a pack assembly including several rechargeable lithium battery cells, excess heat produced in one of the rechargeable lithium battery cells may spread to the adjacent cells in succession, thereby causing the entire module and/or pack to overheat.

### SUMMARY

One or more aspects are directed toward an electrolyte for a rechargeable lithium battery that may suppress or reduce ignition and/or explosion of the corresponding battery cell at relatively high temperatures. In some aspects, the electrolyte may prevent or reduce a temperature increase in the corresponding battery cell even if (e.g., when) an ignition and/or explosion occurs, or begins to occur, in an adjacent battery cell.

Additional aspects will be set forth in part in the description which follows and, in part, will be apparent from the description, or may be learned by practice of the presented embodiments of the disclosure.

According to one or more embodiments an electrolyte for a rechargeable lithium battery includes a non-aqueous organic solvent; a lithium salt; a first additive represented by Chemical Formula 1; and a second additive represented by Chemical Formula 2:

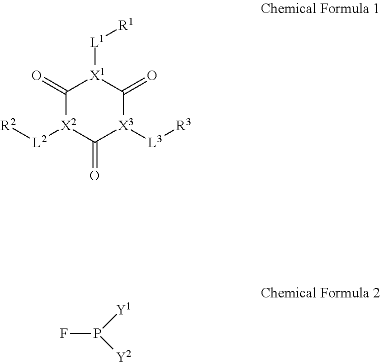

The rechargeable lithium battery of one or more embodiments is capable of suppressing or reducing ignition and/or explosion of the corresponding battery cell at relatively high temperatures by combining the two types (kinds) of additives disclosed herein. In some embodiments, the rechargeable lithium battery prevents the temperature of the corresponding battery cell from (protect from temperature) increasing even if (e.g. when) an ignition and/or explosion occurs, or begins to occur, in an adjacent battery cell.

### BRIEF DESCRIPTION OF THE DRAWING

The drawing is a schematic view showing a rechargeable lithium battery according to one or more embodiments.

### DETAILED DESCRIPTION

Hereinafter, a rechargeable lithium battery according to one or more embodiments will be described in more detail with reference to the accompanying drawing, so that those of ordinary skill in the art can easily implement them. Examples of the embodiments are illustrated and described by referring to the accompanying drawing to explain aspects of the present description. However, these embodiments are merely examples, and the present disclosure is not limited thereto. Rather the present disclosure may be embodied in many different forms and is defined by the scope of claims.

The terminology utilized herein is utilized to describe embodiments only, and is not intended to limit the present disclosure. The singular expressions “a,” “an,” and “the” include the plural expressions, including “at least one,” unless the context clearly dictates otherwise.

As utilized herein, “combination thereof” refers to a mixture, laminate, composite, copolymer, alloy, blend, reaction product, and/or the like of the constituents.

Herein, it should be understood that terms such as “comprises,” “comprise,” “comprising,” “includes,” “including,” “include,” “having,” “has,” and/or “have” are intended to designate the presence of an embodied aspect, number, operation, element, and/or any suitable combination thereof, but it does not preclude the possibility of the presence or addition of one or more other feature, number, operation, element, and/or any suitable combination thereof.

In the drawing, the thickness of layers, films, panels, regions, and/or the like, are exaggerated for clarity wherein like reference numerals designate like elements, and duplicative descriptions thereof may not be provided throughout the specification. It will be understood that if (e.g., when) an element such as a layer, film, region, or substrate is referred to as being “on” another element, it can be directly on the other element or intervening elements may also be present. In contrast, if (e.g., when) an element is referred to as being “directly on” another element, there are no intervening elements present.

In one or more embodiments, the term “layer” herein includes not only a shape formed on the whole surface if (e.g., when) viewed from a plan view, but also a shape formed on a partial surface.

It will be understood that, although the terms “first,” “second,” “third,” and/or the like may be utilized herein to describe one or more suitable elements, components, regions, layers and/or sections, these elements, components, regions, layers and/or sections should not be limited by these terms. These terms are only utilized to distinguish one element, component, region, layer or section from another element, component, region, layer, or section. Thus, a first element, component, region, layer, or section described herein may be termed a second element, component, region, layer or section without departing from the teachings set forth herein.

As utilized herein, the term “and/or” includes any, and all, combinations of one or more of the associated listed items. Expressions such as “at least one of,” “one of,” and “selected from,” if (e.g., when) preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

Spatially relative terms, such as “beneath,” “below,” “lower,” “above,” “upper” and/or the like, may be utilized herein to easily describe the relationship between one element or feature and another element or feature. It will be understood that the spatially relative terms are intended to encompass different orientations of a device in utilization or operation in addition to the orientation illustrated in the drawings. For example, if (e.g., when) the device in the drawing is turned over, elements described as “below” or “beneath” other elements or features will be oriented “above” the other elements or features. Thus, the example term “below” can encompass both (e.g., simultaneously) the orientations of above and below. The device may be otherwise oriented (rotated 90 degrees or at other orientations), and the spatially relative terms utilized herein may be interpreted accordingly.

The terminology utilized herein is utilized for the purpose of describing particular embodiments only, and is not intended to limit the present disclosure. Unless otherwise defined, all terms (including chemical, technical and scientific terms) utilized herein have the same meaning as commonly understood by one of ordinary skill in the art to which this disclosure pertains. It will be further understood that terms, such as those defined in commonly utilized dictionaries, should be interpreted as having a meaning that is consistent with their meaning in the context of the related art and the present disclosure, and will not be interpreted in an idealized or overly formal sense.

Example embodiments are described herein with reference to a schematic view of idealized embodiments. As such, variations from the shapes of the illustrations as a result, for example, of manufacturing techniques and/or tolerances, are to be expected. Thus, embodiments described herein should not be construed as being limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the drawing are schematic in nature and their shapes are not intended to illustrate the precise shape of a region and are not intended to limit the scope of the present claims.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

In this context, “consisting essentially of” means that any additional components will not materially affect the chemical, physical, optical or electrical properties of the semiconductor film.

Further, in this specification, the phrase “on a plane,” or “plan view,” means viewing a target portion from the top, and the phrase “on a cross-section” means viewing a cross-section formed by vertically cutting a target portion from the side.

### Definitions

The term “particle diameter” as utilized herein refers to an average diameter of particles if (e.g., when) the particles are spherical, and refers to an average major axis length of particles if (e.g., when) the particles are non-spherical. For example, the average particle diameter may be measured by a method well suitable to those skilled in the art, for example, may be measured by a particle size analyzer, or may be measured by a transmission electron microscopic image or a scanning electron microscopic image. It may be possible to obtain an average particle diameter value by measuring utilizing a dynamic light scattering method, performing data analysis, counting the number of particles for each particle size range, and calculating from this. Unless otherwise defined, the average particle diameter may refer to the diameter (D₅₀) of particles having a cumulative volume of 50 volume % in the particle size distribution. If (e.g., when) measuring by laser diffraction, more specifically, the particles to be measured are dispersed in a dispersion medium and then introduced into a related art laser diffraction particle size measuring device (e.g., MT 3000 available from Microtrac, Ltd.) utilizing ultrasonic waves at about 28 kHz, and after irradiation with an output of 60 W, the average particle diameter (D50) based on 50% of the particle size distribution in the measuring device can be calculated. As utilized herein, if (e.g., when) a definition is not otherwise provided, the average particle diameter refers to a diameter (D₅₀) of particles having a cumulative volume of 50 volume % in the particle size distribution that is obtained by measuring the size (diameter or length of the major axis) of about 20 particles at random in a scanning electron microscopic image.

In present disclosure, “not include a or any ‘component’” “exclude a or any ‘component”’, “‘component’-free”, and/or the like refers to that the “component” not being added, selected or utilized as a component in the composition/structure, but the “component” of less than a suitable amount may still be included due to other impurities and/or external factors.

Herein, “or” is not to be construed as an exclusive meaning, for example, “A or B” is construed to include A, B, A+B, and/or the like.

“Metal” is interpreted as a concept including ordinary metals, transition metals and metalloids (semi-metals).

As utilized herein, if (e.g., when) specific definition is not otherwise provided, “substituted” refers to replacement of at least one hydrogen of a compound by a halogen atom (F, Cl, Br, or I), a hydroxy group, a C1 to C20 alkoxy group, a nitro group, a cyano group, an amine group, an imino group, an azido group, an amidino group, a hydrazino group, a hydrazono group, a carbonyl group, a carbamyl group, a thiol group, an ester group, an ether group, a carboxyl group or a salt thereof, a sulfonic acid group or a salt thereof, a phosphoric acid or a salt thereof, a C1 to C20 alkyl group, a C2 to C20 alkenyl group, a C2 to C20 alkynyl group, a C6 to C30 aryl group, a C3 to C20 cycloalkyl group, a C3 to C20 cycloalkenyl group, a C3 to C20 cycloalkynyl group, a C2 to C20 heterocycloalkyl group, a C2 to C20 heterocycloalkenyl group, a C2 to C20 heterocycloalkynyl group, and/or a (e.g., any suitable) combination thereof.

As utilized herein, if (e.g., when) specific definition is not otherwise provided, “heterocycloalkyl group”, “heterocycloalkenyl group”, “heterocycloalkynyl group,” and “heterocycloalkylene group” refer to presence of at least one N, O, S, or P in a cyclic compound of cycloalkyl, cycloalkenyl, cycloalkynyl, and cycloalkylene.

In the chemical formula of the present specification, unless a specific definition is otherwise provided, hydrogen is bonded at the position if (e.g., when) a chemical bond is not drawn where supposed to be given.

As utilized herein, if (e.g., when) a definition is not otherwise provided, “*” refers to a linking part between the same or different atoms, or chemical formulas.

### Electrolyte

Some embodiments provide an electrolyte for a rechargeable lithium battery including a non-aqueous organic solvent; a lithium salt; a first additive represented by Chemical Formula 1; and a second additive represented by Chemical Formula 2:

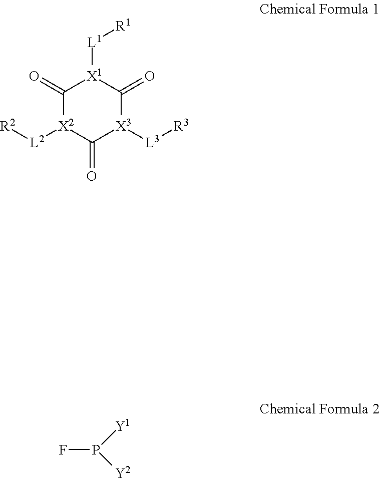

The first additive has the aspect of gelling the electrolyte at relatively high temperatures. The second additive has the aspect of delaying structural collapse of the positive electrode surface at relatively high temperatures by binding to the positive electrode transition metal oxide (e.g., as a ligand).

Overall, the combination of the two types (kinds) of additives described herein has the aspect of rapidly increasing the viscosity of the electrolyte (e.g., at relatively high temperatures) and, at the same time, drastically reducing ionic conductivity. For example, the combined properties may thereby disconnect (e.g., shut down) the rechargeable lithium battery cell (e.g., from the surroundings, such as other battery cells).

Accordingly, a rechargeable lithium battery in which the electrolyte for a rechargeable lithium battery of one or more embodiments is utilized or applied can suppress or reduce ignition and/or explosion of the cell at relatively high temperature by a combination of the two types (kinds) of additives described herein, and even if (e.g., when) ignition and/or explosion starts in an adjacent cell, the temperature increase in that cell can be prevented or reduced.

Hereinafter, an electrolyte for a rechargeable lithium battery of one or more embodiments will be described in more detail.

### First Additive

The description of Chemical Formula 1 representing the first additive is as follows.
X¹ to X³ are each N or CH, provided that at least one of (e.g., selected from among) X¹ to X³ is N.

In one or more embodiments, X¹ to X³ may each (e.g., all) be N.
L¹ to L³ may each independently be a single bond, or a substituted or unsubstituted C1 to C10 alkylene group.

In one or more embodiments, L¹ to L³ may each independently be a substituted or unsubstituted C1 to C5 alkylene group.
R¹ to R³ may each independently be an epoxy group, or a substituted or unsubstituted C1 to C10 alkyl group, provided that at least one of (e.g., selected from among) R¹ to R³ is epoxy group.

In one or more embodiments, R¹ to R³ may each (e.g., all) be an epoxy group.

In one or more embodiments, the first additive (e.g., Chemical Formula 1) may be represented by Chemical Formula 1-1:

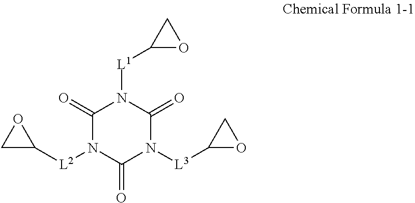

In Chemical Formula 1-1,
L¹ to L³ may each independently be a substituted or unsubstituted C1 to C5 alkylene group.

Representative examples of the first additive are as follows:

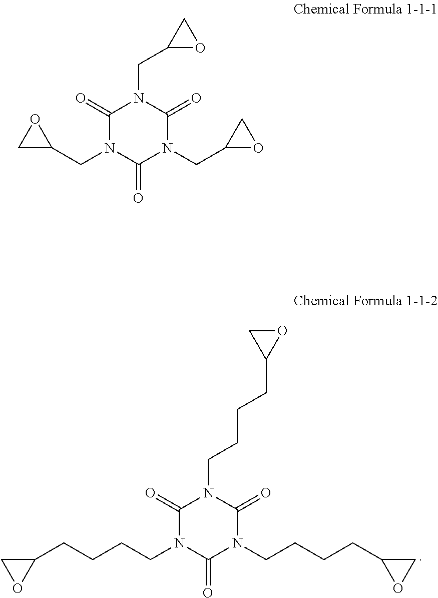 [SMILES: O=c1n(CC2CO2)c(=O)n(CC2CO2)c(=O)n1CC1CO1 ; O=c1n(CCCCC2CO2)c(=O)n(CCCCC2CO2)c(=O)n1CCCCC1CO1]

### Second Additive

The description of Chemical Formula 2 representing the second additive is as follows.
Y¹ and Y² may each independently be a halogen group or —O-L⁴-R⁴, provided that at least one of (e.g., selected from among) Y¹ and Y² is —O-L⁴-R⁴.
L⁴ may be a single bond or a substituted or unsubstituted C1 to C10 alkylene group.
R⁴ may be a cyano group (—CN), a difluorophosphite group (—OPF₂), a substituted or unsubstituted C1 to C10 alkyl group, a substituted or unsubstituted C2 to C10 alkenyl group, a substituted or unsubstituted C3 to C10 cycloalkyl group, a substituted or unsubstituted C3 to C10 cycloalkenyl group, a substituted or unsubstituted C2 to C10 alkynyl group, a substituted or unsubstituted C3 to C10 cycloalkynyl group, or a substituted or unsubstituted C6 to C20 aryl group.

If (e.g., when) Y¹ and Y² are concurrently (e.g., simultaneously) —O-L⁴-R⁴, two R⁴s may not be linked (e.g., exists independently) or two R⁴s may be linked to form (or provide) a substituted or unsubstituted monocyclic or polycyclic aliphatic heterocycle, or a substituted or unsubstituted monocyclic or polycyclic aromatic heterocycle.

In one or more embodiments, one selected from among (e.g., either) Y¹ and Y² may be a fluoro atom and the other one (e.g., one selected from among Y¹ and Y²), may be —O-L⁵-R⁵; wherein L⁵ may be a single bond or a substituted or unsubstituted C1 to C10 alkylene group; and R⁵ may be a cyano group (—CN) or a difluorophosphite group (—OPF₂).

In one or more embodiments, Y¹ may be —O-L⁶-R⁶ and Y² may be O-L⁷-R⁷; wherein L⁶ and L⁷ may each independently be a single bond or a substituted or unsubstituted C1 to C10 alkylene group; R⁶ and R⁷ may each independently be a substituted or unsubstituted C1 to C10 alkyl group, and R⁶ and R⁷ may be linked to form (or provide) a substituted or unsubstituted monocyclic or polycyclic aliphatic heterocycle.

In one or more embodiments, the second additive (e.g., Chemical Formula 2) may be represented by Chemical Formula 2-1 or 2-2:

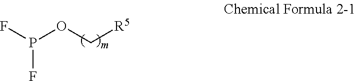

wherein, in Chemical Formula 1-1,
m may be an integer of 1 to 5; and
R⁵ may be a cyano group (—CN) or a difluorophosphite group (—OPF₂).

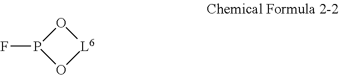

wherein, in Chemical Formula 1-2,
L⁶ may be a substituted or unsubstituted C2 to C5 alkylene group.

In one or more embodiments, the second additive (e.g., Chemical Formula 2-2) may be represented by Chemical Formula 2-2a or 2-2b:

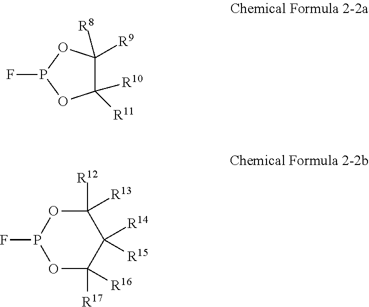

wherein, in Chemical Formulas 2-2a and 2-2b,
R⁸ to R¹⁷ may each independently be a hydrogen atom, a halogen atom, or a substituted or unsubstituted C1 to C5 alkyl group.

Representative examples of the second additive are as follows:

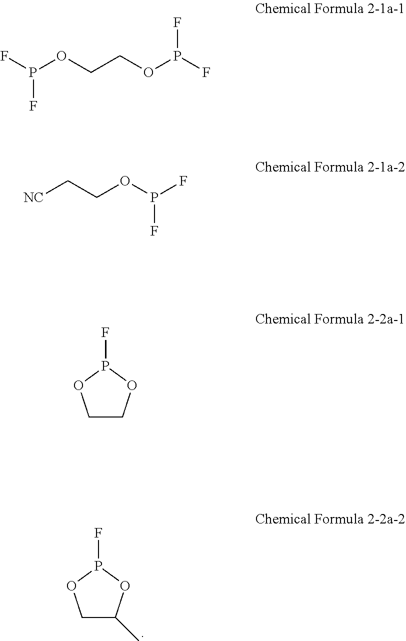

### Contents of First Additive and Second Additive

In one or more embodiments, a weight ratio of the first additive and the second additive may be about 2:1 to about 100:1, or about 3.5:1 to about 70:1.

In one or more embodiments, a sum of the first additive and the second additive may be included in an amount of about 5 to about 30 wt %, or about 7.1 to about 21 wt % based on a total amount of the electrolyte.

In one or more embodiments, the first additive may be included in an amount of about 5 to about 20 wt % based on a total amount of the electrolyte. Additionally, in one or more embodiments, the second additive may be included in an amount of about 0.1 to about 2 wt % based on a total amount of the electrolyte.

In the disclosed ranges, there may be a synergistic aspect due to the combination of the two types (kinds) of additives described herein.

### Non-Aqueous Organic Solvent

The non-aqueous organic solvent serves as a medium for transmitting ions taking part in the electrochemical reaction of a battery.

The non-aqueous organic solvent may include a carbonate-based, ester-based, ether-based, ketone-based, alcohol-based, or aprotic solvent, and/or a (e.g., any suitable) combination thereof.

The carbonate-based solvent may include dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), butylene carbonate (BC), and/or the like. The ester-based solvent may include methyl acetate, ethyl acetate, n-propyl acetate, dimethylacetate, methylpropionate, ethylpropionate, decanolide, mevalonolactone, valerolactone, caprolactone, and/or the like. The ether-based solvent may include dibutyl ether, tetraglyme, diglyme, dimethoxyethane, 2-methyltetrahydrofuran, 2,5-dimethyltetrahydrofuran, tetrahydrofuran, and/or the like. The ketone-based solvent may include cyclohexanone and/or the like. The alcohol-based solvent may include ethanol, isopropyl alcohol, and/or the like, and the aprotic solvent may include nitriles such as R—CN (wherein R is a C2 to C20 linear, branched, or cyclic hydrocarbon group, a double bond, an aromatic ring, or an ether group), amides such as dimethylformamide, and/or the like; dioxolanes such as 1,3-dioxolane, 1,4-dioxolane, and/or the like; sulfolanes, and/or the like.

The non-aqueous organic solvent may be utilized alone or in combination with one or more (e.g., of them), and if (e.g., when) utilized in combination with one or more, a mixing ratio may be appropriately or suitably adjusted according to the desired or suitable battery performance, which is well understood by those skilled in the art.

Additionally, if (e.g., when) utilizing a carbonate-based solvent, a cyclic carbonate and a chain carbonate may be mixed and utilized. For example, the cyclic carbonate and the chain carbonate may be mixed at a volume ratio of about 1:1 to about 1:9.

The non-aqueous organic solvent may further include an aromatic hydrocarbon-based organic solvent. For example, a carbonate-based solvent and an aromatic hydrocarbon-based organic solvent may be mixed and utilized in a volume ratio of about 1:1 to about 30:1.

The electrolyte may further include vinylethyl carbonate, vinylene carbonate, or an ethylene carbonate-based compound to improve battery cycle-life.

Representative examples of the ethylene carbonate-based compound may include fluoroethylene carbonate, difluoroethylene carbonate, chloroethylene carbonate, dichloroethylene carbonate, bromoethylene carbonate, dibromoethylene carbonate, nitroethylene carbonate, cyanoethylene carbonate, and/or the like.

### Lithium Salt

The lithium salt is dissolved in an organic solvent, supplies a battery with lithium ions, enables the general operation of the rechargeable lithium battery, and improves transportation of the lithium ions between positive and negative electrodes. Examples of the lithium salt may include at least one selected from among LiPF₆, LiBF₄, LiSbF₆, LiAsF₆, LiClO₄, LiAlO₂, LiAlCl₄, LiPO₂F₂, LiCl, Lil, LiN(SO₃C₂F₅)₂, Li(FSO₂)₂N (lithium bis(fluorosulfonyl)imide, LiFSI), LiC₄F₉SO₃, LiN(CₓF₂ₓ₊₁SO₂)(C_{y}F_{2y+1}SO₂), x and y are integers in a range of 1 to 20, lithium trifluoromethane sulfonate, lithium tetrafluoroethane sulfonate, lithium difluorobis(oxalato)phosphate (LiDFOB), lithium bis(oxalato) borate (LiBOB).

A concentration of the lithium salt may be utilized within the range of about 0.1 M to about 2.0 M. If (e.g., when) the concentration of the lithium salt is within the described range, the electrolyte has appropriate or suitable conductivity and viscosity, and thus excellent or suitable electrolyte performance can be exhibited, and lithium ions can move effectively.

### Rechargeable Lithium Battery

In one or more embodiments, a rechargeable lithium battery includes a positive electrode; a negative electrode; and the electrolyte according to one or more of the aforementioned embodiments.

The rechargeable lithium battery of one or more embodiments includes the additive of the aforementioned embodiment, or the electrolyte of the aforementioned embodiment, thereby suppressing or reducing ignition and/or explosion of the corresponding cell at relatively high temperatures, and preventing or reducing temperature increase in the corresponding cell even if (e.g., when) ignition and/or explosion begins in an adjacent cell.

Hereinafter, descriptions that overlap with the preceding disclosure will not be provided, and the rechargeable lithium battery will be described in more detail.

### Positive Electrode Active Material

The positive electrode active material may be a compound capable of reversibly intercalating and deintercalating lithium (a lithiated intercalation compound). For example, one or more types (kinds) of composite oxides of lithium and a metal selected from among cobalt, manganese, nickel, and/or a (e.g., any suitable) combination thereof may be utilized.

The composite oxide may be a lithium transition metal composite oxide, and specific examples may include lithium nickel-based oxide, lithium cobalt-based oxide, lithium manganese-based oxide, lithium iron phosphate-based compound, cobalt-free lithium nickel-manganese-based oxide, and/or a (e.g., any suitable) combination thereof.

As an example, the positive electrode active material may be a relatively high nickel-based positive electrode active material having a nickel content (e.g., amount) of about 80 mol % or more based on 100 mol % of metals excluding lithium in the lithium transition metal composite oxide. The nickel content (e.g., amount) in the relatively high nickel-based positive electrode active material may be about 85 mol % or more, about 90 mol % or more, about 91 mol % or more, or about 94 mol % or more and about 99 mol % or less based on 100 mol % of metals excluding lithium. High-nickel-based positive electrode active materials can achieve relatively high capacity and can be applied to a relatively high-capacity, relatively high-density rechargeable lithium battery.

As a more specific example, a compound represented by any of the following chemical formulas may be utilized. LiₐA_{1-b}X_{b}O_{2-c}D_{c} (0.90≤a≤1.8, 0≤b≤0.5, 0≤c≤0.05); LiₐMn_{2-b}X_{b}O_{4-c}D_{c} (0.90≤a≤1.8, 0≤b≤0.5, 0≤c≤0.05); LiₐNi_{1-b-c}CO_{b}X_{c}O_{2-α}D_{α} (0.90≤a≤1.8, 0≤b≤0.5, 0≤c≤0.5, 0<α<2); LiₐNi_{1-b-c}Mn_{b}X_{c}O_{2-α}D_{α} (0.90≤a≤1.8, 0≤b≤0.5, 0≤c≤0.5, 0<α<2); LiₐNi_{b}Co_{c}L¹_{d}GₑO₂(0.90≤a≤1.8, 0≤b≤0.9, 0≤c≤0.5, 0≤d≤0.5, 0≤e≤0.1); LiₐNiG_{b}O₂ (0.90≤a≤1.8, 0.001≤b≤0.1); LiₐCoG_{b}O₂ (0.90≤a≤1.8, 0.001≤b≤0.1); LiₐMn_{1-b}G_{b}O₂ (0.90≤a≤1.8, 0.001≤b≤0.1); LiₐMn₂G_{b}O₄ (0.90≤a≤1.8, 0.001≤b≤0.1); LiₐMn_{1-g}G_{g}PO₄ (0.90≤a≤1.8, 0≤g≤0.5); Li_{(3-f)}Fe₂(PO₄)₃ (0≤f≤2); LiₐFePO₄ (0.90≤a≤1.8)

In the preceding chemical formulas, A is Ni, Co, Mn, and/or a (e.g., any suitable) combination thereof; X is Al, Ni, Co, Mn, Cr, Fe, Mg, Sr, V, a rare earth element, and/or a (e.g., any suitable) combination thereof; D is O, F, S, P, and/or a (e.g., any suitable) combination thereof; G is Al, Cr, Mn, Fe, Mg, La, Ce, Sr, V, and/or a (e.g., any suitable) combination thereof; Q is Ti, Mo, Mn, and/or a (e.g., any suitable) combination thereof; Z is Cr, V, Fe, Sc, Y, and/or a (e.g., any suitable) combination thereof; and L¹ is Mn, Al, and/or a (e.g., any suitable) combination thereof.

### Positive Electrode

The positive electrode for a rechargeable lithium battery may include a current collector and a positive electrode active material layer formed on the current collector. The positive electrode active material layer includes a positive electrode active material and may further include a binder and/or a conductive material.

A content (e.g., amount) of the positive electrode active material may be about 90 wt % to about 99.5 wt % based on 100 wt % of the positive electrode active material layer.

In some example embodiments, the positive electrode active material layer may further include a binder and a conductive material. At this time, each content (e.g., amount) of the binder and the conductive material may be about 0.5 wt % to about 5 wt % based on 100% by weight of the positive electrode active material layer.

The binder serves to ensure that the positive electrode active material particles adhere to each other and also to adhere the positive electrode active material to the current collector. Examples of the binder may include polyvinyl alcohol, carboxymethyl cellulose, hydroxypropyl cellulose, diacetyl cellulose, polyvinyl chloride, carboxylated polyvinyl chloride, polyvinyl fluoride, an ethylene oxide-containing polymer, polyvinylpyrrolidone, polyurethane, polytetrafluoroethylene, polyvinylidene fluoride, polyethylene, polypropylene, a styrene-butadiene rubber, a (meth)acrylated styrene-butadiene rubber, an epoxy resin, a (meth)acrylic resin, a polyester resin, and nylon, but are not limited thereto.

The conductive material is utilized to impart conductivity to the electrode, and in the battery being configured, any electronically conductive material can be utilized as long as it does not cause chemical change. Examples of the conductive material may include a carbon-based material such as natural graphite, artificial graphite, carbon black, acetylene black, ketjen black, a carbon fiber, a carbon nanofiber, and a carbon nanotube; a metal-based material including copper, nickel, aluminum, silver, and/or the like, and in the form of (or provide) a metal powder or a metal fiber; a conductive polymer such as a polyphenylene derivative; and/or a (e.g., any suitable) mixture thereof.

The current collector may include Al, but the present disclosure is not limited thereto.

### Negative Electrode Active Material

The negative electrode active material may be a material that reversibly intercalates/deintercalates lithium ions, a lithium metal, a lithium metal alloy, a material capable of doping and dedoping lithium, or a transition metal oxide.

The material that reversibly intercalates/deintercalates lithium ions may be a carbon-based negative electrode active material, for example crystalline carbon, amorphous carbon and/or a (e.g., any suitable) combination thereof. Examples of the crystalline carbon may include graphite such as irregular-shaped, plate-shaped, flake, spherical or fibrous natural graphite or artificial graphite, and examples of the amorphous carbon may include soft carbon or hard carbon, a mesophase pitch carbonized product, calcined coke, and/or the like.

The lithium metal alloy may include an alloy including lithium and a metal selected from among Na, K, Rb, Cs, Fr, Be, Mg, Ca, Sr, Si, Sb, Pb, In, Zn, Ba, Ra, Ge, Al, and Sn.

A Si-based negative electrode active material or a Sn-based negative electrode active material may be utilized as a material capable of doping and dedoping lithium. The Si-based negative electrode active material may include silicon, a silicon-carbon composite, SiOx (0<x≤2), a Si-Q alloy (wherein Q is an element selected from among an alkali metal, an alkaline earth metal, a Group 13 element, Group 14 element (excluding Si), Group 15 element, Group 16 element, a transition metal, a rare earth element, and/or a (e.g., any suitable) combination thereof, for example Mg, Ca, Sr, Ba, Ra, Sc, Y, Ti, Zr, Hf, Rf, V, Nb, Ta, Db, Cr, Mo, W, Sg, Tc, Re, Bh, Fe, Pb, Ru, Os, Hs, Rh, Ir, Pd, Pt, Cu, Ag, Au, Zn, Cd, B, Al, Ga, Sn, In, TI, Ge, P, As, Sb, Bi, S, Se, Te, Po, and/or a (e.g., any suitable) combination thereof), and/or a (e.g., any suitable) combination thereof. The Sn-based negative electrode active material may be Sn, SnO₂, SnOₓ (0<x<2), a Sn alloy, and/or a (e.g., any suitable) combination thereof.

The silicon-carbon composite may be a composite of silicon and amorphous carbon. An average particle diameter (D₅₀) of the silicon-carbon composite particles may be, for example, about 0.5 micrometer (μm) to about 20 μm. According to some example embodiments, the silicon-carbon composite may be in the form of (or provide) silicon particles and amorphous carbon coated on the surface of the silicon particles. For example, it may include a secondary particle (core) in which silicon primary particles are assembled (agglomerated) and an amorphous carbon coating layer (shell) on the surface of the secondary particle. The amorphous carbon may also be arranged between the silicon primary particles, and for example, the silicon primary particles may be coated with amorphous carbon. The secondary particles may be (exist) dispersed in an amorphous carbon matrix.

The silicon-carbon composite may further include crystalline carbon. For example, the silicon-carbon composite may include a core including crystalline carbon and silicon particles and an amorphous carbon coating layer on the surface of the core. The crystalline carbon may be artificial graphite, natural graphite, and/or a (e.g., any suitable) combination thereof. The amorphous carbon may include soft carbon or hard carbon, a mesophase pitch carbonized product, and calcined coke.

If (e.g., when) the silicon-carbon composite includes silicon and amorphous carbon, a content (e.g., amount) of silicon may be about 10 wt % to about 50 wt %, and a content (e.g., amount) of amorphous carbon may be about 50 wt % to about 90 wt % based on 100 wt % of the silicon-carbon composite. In one or more embodiments, if (e.g., when) the composite includes silicon, amorphous carbon, and crystalline carbon, a content (e.g., amount) of silicon may be about 10 wt % to about 50 wt %, and a content (e.g., amount) of crystalline carbon may be about 10 wt % to about 70 wt %, and a content (e.g., amount) of amorphous carbon may be about 20 wt % to about 40 wt % based on 100 wt % of the silicon-carbon composite.

Additionally, a thickness of the amorphous carbon coating layer may be about 5 nanometer (nm) to about 100 nm. An average particle diameter (D₅₀) of the silicon particles (primary particles) may be about 10 nm to about 1 μm, or about 10 nm to about 200 nm. The silicon particles may exist as silicon alone, in the form of (or provide) a silicon alloy, or in an oxidized form. The oxidized form of silicon may be represented by SiOₓ (0<x≤2). At this time, an atomic content (e.g., amount) ratio of Si:O, which indicates a degree of oxidation, may be about 99:1 to about 33:67. In the present specification, as utilized herein, if (e.g., when) a definition is not otherwise provided, an average particle diameter (D₅₀) indicates a particle where an accumulated volume is about 50 vol % in a particle size distribution.

The Si-based negative electrode active material or Sn-based negative electrode active material may be utilized by mixing with a carbon-based negative electrode active material. If (e.g., when) utilizing a mixture of Si-based negative electrode active material or Sn-based negative electrode active material and carbon-based negative electrode active material, a mixing ratio may be about 1:99 to about 90:10 by weight.

### Negative Electrode

A negative electrode for a rechargeable lithium battery includes a current collector and a negative electrode active material layer on the current collector. The negative electrode active material layer includes a negative electrode active material and may further include a binder and/or a conductive material.

A content (e.g., amount) of the negative electrode active material may be about 95 wt % to about 99.5 wt % based on 100 wt % of the negative electrode active material layer. A content (e.g., amount) of the binder may be about 0.5 wt % to about 5 wt % based on 100 wt % of the negative electrode active material layer. A content (e.g., amount) of the binder may be about 0.5 wt % to about 5 wt % based on 100 wt % of the negative electrode active material layer. For example, the negative electrode active material layer may include about 90 wt % to about 99 wt % of the negative electrode active material, about 0.5 wt % to about 5 wt % of the binder, and about 0.5 wt % to about 5 wt % of the conductive material.

The binder serves to adhere the negative electrode active material particles to each other and also helps the negative electrode active material to adhere to the current collector. The binder may be a non-aqueous binder, an aqueous binder, a dry binder, and/or a (e.g., any suitable) combination thereof.

The non-aqueous binder may include polyvinyl chloride, carboxylated polyvinyl chloride, polyvinyl fluoride, an ethylene propylene copolymer, polystyrene, polyurethane, polytetrafluoroethylene, polyvinylidene fluoride, polyethylene, polypropylene, polyamide-imide, polyimide, and/or a (e.g., any suitable) combination thereof.

The aqueous binder may be (e.g., may be selected from among) a styrene-butadiene rubber, a (meth)acrylated styrene-butadiene rubber, a (meth)acrylonitrile-butadiene rubber, a (meth)acrylic rubber, a butyl rubber, a fluorine rubber, polyethylene oxide, polyvinylpyrrolidone, polyepichlorohydrine, polyphosphazene, poly(meth)acrylonitrile, an ethylene propylene diene copolymer, polyvinylpyridine, chlorosulfonated polyethylene, latex, a polyester resin, a (meth)acrylic resin, a phenol resin, an epoxy resin, polyvinyl alcohol, and/or a (e.g., any suitable) combination thereof.

If (e.g., when) an aqueous binder is utilized as the negative electrode binder, it may further include a cellulose-based compound capable of imparting viscosity. As this cellulose-based compound, one or more types (kinds) of carboxymethyl cellulose, hydroxypropylmethyl cellulose, methyl cellulose, or an alkali metal salt thereof may be utilized. The alkali metal may be Na, K, or Li.

The dry binder may be a polymer material capable of being fiberized, and may be, for example, polytetrafluoroethylene, polyvinylidene fluoride, a polyvinylidene fluoride-hexafluoropropylene copolymer, polyethylene oxide, and/or a (e.g., any suitable) combination thereof.

The conductive material is utilized to impart conductivity to the electrode, and in the battery being configured, any electronically conductive material can be utilized as long as it does not cause chemical change. Specific examples may include carbon-based materials such as natural graphite, artificial graphite, carbon black, acetylene black, ketjen black, a carbon fiber, a carbon nanofiber, and a carbon nanotube; a metal-based material including copper, nickel, aluminum, silver, and/or the like, in the form of (or provide) a metal powder or a metal fiber; a conductive polymer such as a polyphenylene derivative; and/or a (e.g., any suitable) mixture thereof.

The negative electrode current collector may be of (e.g., may be selected from among) a copper foil, a nickel foil, a stainless steel foil, a titanium foil, a nickel foam, a copper foam, a polymer substrate coated with a conductive metal, and/or a (e.g., any suitable) combination thereof.

### Separator

The rechargeable lithium battery may further include a separator between the negative electrode and the positive electrode, depending on a type or kind of the rechargeable lithium battery. Such a separator may be for example may include polyethylene, polypropylene, polyvinylidene fluoride, and multi-layers thereof such as a polyethylene/polypropylene double-layered separator, a polyethylene/polypropylene/polyethylene triple-layered separator, and a polypropylene/polyethylene/polypropylene triple-layered separator.

The separator may include a porous substrate and a coating layer containing an organic material, an inorganic material, and/or a (e.g., any suitable) combination thereof on a (e.g., one or both surfaces or sides (e.g., opposite surfaces)) of the porous substrate.

The porous substrate may be a polymer film formed of any one selected from among polyolefin such as polyethylene and polypropylene, polyester such as polyethylene terephthalate and polybutylene terephthalate, polyacetal, polyamide, polyimide, polycarbonate, polyether ketone, polyaryl ether ketone, polyetherimide, polyamideimide, polybenzimidazole, polyethersulfone, polyphenylene oxide, a cyclic olefin copolymer, polyphenylene sulfide, polyethylene naphthalate, a glass fiber, Teflon, and polytetrafluoroethylene, or a copolymer and/or a (e.g., any suitable) mixture of two or more of them.

The porous substrate may have a thickness of about 1 μm to about 40 μm, for example about 1 μm to about 30 μm, about 1 μm to about 20 μm, about 5 μm to about 15 μm, or about 10 μm to about 15 μm.

The organic material may include a (meth)acryl-based copolymer including a first structural unit derived from (meth)acrylamide, and a second structural unit including at least one of a structural unit derived from (meth)acrylic acid or (meth)acrylate, and a structural unit derived from (meth)acrylamidosulfonic acid or a salt thereof.

The inorganic material may include inorganic particles selected from among Al₂O₃, SiO₂, TiO₂, SnO₂, CeO₂, MgO, NiO, CaO, GaO, ZnO, ZrO₂, Y₂O₃, SrTiO₃, BaTiO₃, Mg(OH)₂, boehmite, and/or a (e.g., any suitable) combination thereof, but the present disclosure is not limited thereto. An average particle diameter (D₅₀) of the inorganic particles may be about 1 nm to about 2000 nm, for example, about 100 nm to about 1000 nm, or about 100 nm to about 700 nm.

The organic material and the inorganic material may be mixed in one coating layer or may exist in a stacked form of (or provide) a coating layer including an organic material and a coating layer including an inorganic material.

A thickness of the coating layer may be each about 0.5 μm to about 20 μm, for example, about 1 μm to about 10 μm, or about 1 μm to about 5 μm.

### Rechargeable Lithium Battery

The rechargeable lithium battery may be classified into cylindrical, prismatic, pouch, coin, and/or the like, depending on their shape. The drawing is a schematic view showing a rechargeable lithium battery according to some example embodiments. Referring to the drawing, the rechargeable lithium battery 100 includes an electrode assembly 40 including a separator 30 interposed between the positive electrode 10 and the negative electrode 20, and a case 50 in which the electrode assembly 40 is housed. The positive electrode 10, the negative electrode 20, and the separator 30 may be impregnated with an electrolyte. The rechargeable lithium battery 100 may include a sealing member 60 that seals the case 50 as shown in the drawing.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

Hereinafter, examples and comparative examples of the present disclosure will be described. However, the present disclosure is not limited by the following examples.

### EXAMPLES

### Examples 1 to 8 and Comparative Examples 1 to 5

### (1) Preparation of Electrolyte

As the non-aqueous organic solvent, a carbonate-based solvent mixed in a volume ratio of ethylene carbonate (EC):ethylmethyl carbonate (EMC):dimethyl carbonate (DMC)=20:10:70 was utilized.

1.5 M lithium salt (LiPF₆) was mixed with the non-aqueous organic solvent, and additives were added according to the compositions in Table 1 to obtain electrolytes.

Chemical Formula 1-1-1] (Tris (2,3-epoxypropyl) isocyanurate, CAS No. 2451-62-9

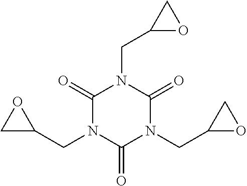 [SMILES: O=c1n(CC2CO2)c(=O)n(CC2CO2)c(=O)n1CC1CO1]

Chemical Formula 1-1-2] (1,3,5-tris[4-(2-oxiranyl)butyl]-1,3,5-triazine-2,4,6-trione, CAS No. 91403-65-5

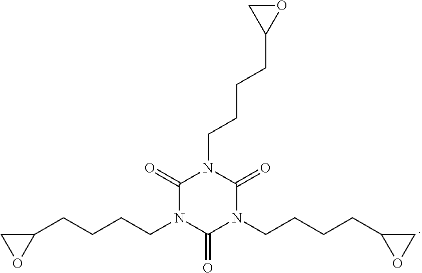 [SMILES: O=c1n(CCCCC2CO2)c(=O)n(CCCCC2CO2)c(=O)n1CCCCC1CO1]

Chemical Formula 2-1a-1] (CAS No. 3965-00-2 253084/412299

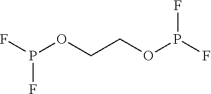

Chemical Formula 2-2a-2] (CAS No. 16415-09-1

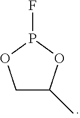

### (2) Manufacturing of Rechargeable Lithium Battery Cells

LiNi_{0.91}Co_{0.04}Al_{0.05}O₂ as a positive electrode active material, polyvinylidene fluoride as a binder, and ketjen black as a conductive material were mixed respectively in a weight ratio of 98.5:0.75:0.75 and then, dispersed in N-methyl pyrrolidone to prepare a positive electrode active material slurry.

The positive electrode active material slurry was coated on a 14 micrometer (μm)-thick Al foil, dried at 110° C., and pressed to manufacture a positive electrode.

A mixture including artificial graphite and silicon particles mixed in a weight ratio of 93:7 was utilized as a negative electrode active material, and the negative electrode active material, a styrene-butadiene rubber binder, and carboxylmethyl cellulose in a weight ratio of 97:1:2 were dispersed in distilled water to prepare a negative electrode active material slurry.

The negative electrode active material slurry was coated on a 10 μm-thick Cu foil, dried at 100° C., and pressed to manufacture a negative electrode.

The manufactured positive and negative electrodes were assembled with a 25 μm-thick polyethylene separator to manufacture an electrode assembly, the electrode assembly was accommodated in a cylindrical can with a width of 21 millimeter (mm) and a length of 70 mm as a battery container, and the electrolyte of the Example 1 was injected thereinto to manufacture a rechargeable lithium battery cell.

TABLE 1

| | First additive | | Second additive | | Sum content |
|---|---|---|---|---|---|
| | | Content | | Content | (e.g., Amount) |
| | Chemical | (e.g., Amount) | Chemical | (e.g., Amount) | of additives |
| | Formula | (wt %) | Formula | (wt %) | (wt %) |
| Comp. | — | — | — | — | — |
| Ex. 1 | | | | | |
| Comp. | Chemical | 7 | — | — | 7 |
| Ex. 2 | Formula 1-1-1 | | | | |
| Comp. | Chemical | 7 | — | — | 7 |
| Ex. 3 | Formula 1-1-2 | | | | |
| Comp. | — | — | Chemical | 1 | 1 |
| Ex. 4 | | | Formula 2-1a-1 | | |
| Comp. | — | — | Chemical | 1 | 1 |
| Ex. 5 | | | Formula 2-2a-2 | | |
| Ex. 1 | Chemical | 7 | Chemical | 1 | 8 |
| | Formula 1-1-1 | | Formula 2-1a-1 | | |
| Ex. 2 | Chemical | 7 | Chemical | 1 | 8 |
| | Formula 1-1-1 | | Formula 2-2a-2 | | |
| Ex. 3 | Chemical | 7 | Chemical | 1 | 8 |
| | Formula 1-1-2 | | Formula 2-1a-1 | | |
| Ex. 4 | Chemical | 7 | Chemical | 1 | 8 |
| | Formula 1-1-2 | | Formula 2-2a-2 | | |
| Ex. 5 | Chemical | 5 | Chemical | 1 | 6 |
| | Formula 1-1-1 | | Formula 2-1a-1 | | |
| Ex. 6 | Chemical | 20 | Chemical | 1 | 21 |
| | Formula 1-1-1 | | Formula 2-1a-1 | | |
| Ex. 7 | Chemical | 7 | Chemical | 0.1 | 7.1 |
| | Formula 1-1-1 | | Formula 2-1a-1 | | |
| Ex. 8 | Chemical | 7 | Chemical | 2 | 9 |
| | Formula 1-1-1 | | Formula 2-1a-1 | | |

In the preceding compositions of electrolytes, a “content (e.g., amount) (wt %)” is based on 100 wt % of the total electrolyte (lithium salt+non-aqueous organic solvent+additives).

### Evaluation Example 1: Evaluation of Heat Exposure Situation

For each rechargeable lithium battery cell according to Examples 1 to 8 and Comparative Examples 1 to 5, the heat exposure properties were evaluated.

The rechargeable lithium battery cells were heated from room temperature at a rate of 5° C./min to reach a temperature of 139° C. or higher, and then left to stand at the achieved temperature for 1 hour. The achieved temperature was evaluated as the “heat exposure test passing temperature.”

TABLE 2

| | Heat exposure test passing temperature (° C.) |
|---|---|
| Comparative Example 1 | 139 |
| Comparative Example 2 | 142 |
| Comparative Example 3 | 142 |
| Comparative Example 4 | 140 |
| Comparative Example 5 | 140 |
| Example 1 | 143 |
| Example 2 | 143 |
| Example 3 | 143 |
| Example 4 | 143 |
| Example 5 | 143 |
| Example 6 | 143 |
| Example 7 | 143 |
| Example 8 | 143 |

### Evaluation Example 2: Evaluation of High-Temperature Storage

### (1) Capacity Retention Rate and Capacity Recovery Rate

Each of the rechargeable lithium battery cells of the examples and the comparative examples was once charged and discharged at 0.33 C and measured with respect to charge and discharge capacity (relatively high-temperature storage).

Each of the rechargeable lithium battery cells of the examples and the comparative examples was charged to SOC100% (to reach State of Charge 100% charge capacity based on 100% of total charge capacity of the battery), stored at 60° C. for 30 days, and discharged to 3.0 V at a constant current of 0.33 C and then, measured with respect to initial discharge capacity.

The cells were recharged to 4.3 V at a constant current of 0.33 C and also, to a current of 0.02 C under the constant voltage and then discharged to 3.0 V at 0.33 C to twice measure discharge capacity. A ratio of the first discharge capacity to the initial discharge capacity was shown as a capacity retention rate (retention capacity), and the second discharge capacity was shown as a capacity recovery rate (recovery capacity).

### (2) DC Resistance Change Rate

Each of the rechargeable lithium battery cells of the examples and the comparative examples was measured with respect to ΔV/ΔI (voltage change/current change) to obtain initial DC internal resistance (DCIR) and then, fully charged (SOC 100%) to its internal maximum energy state and stored at a relatively high temperature of 60° C. for 30 days to measure DC resistance, which were utilized to calculate a DCIR increase rate (%) according to Equation 1, and the results are shown in Table 3.

DCIR increase rate=(DCIR after 30 days/initial DCIR)*100 Equation 1

### (3) Open Circuit Voltage Change Rate

Each of the rechargeable lithium battery cells of the examples and the comparative examples were fully charged to SOC 100% in its internal maximum energy state and stored at a relatively high temperature (55±2° C.) for 90 days. After storing the cells at a relatively high temperature for 90 days, an open circuit voltage (OCV) was calculated according to Equation 2, and the results are shown in Table 3.

Open circuit voltage change rate=(open circuit voltage after×days of relatively high temperature storage/open circuit voltage at the start of storage) Equation 2

TABLE 3

| | Capacity | Capacity | DC | Open circuit |
|---|---|---|---|---|
| | retention | recovery | resistance | voltage |
| | rate | rate | change rate | change rate |
| | (%) | (%) | (%) | (%) |
| Comparative | 81.05 | 85.47 | 153 | −2.04 |
| Example 1 | | | | |
| Comparative | 80.54 | 84.33 | 157 | −2.14 |
| Example 2 | | | | |
| Comparative | 80.77 | 84.41 | 158 | −2.18 |
| Example 3 | | | | |
| Comparative | 85.13 | 86.16 | 146 | −1.87 |
| Example 4 | | | | |
| Comparative | 84.93 | 86.17 | 145 | −1.83 |
| Example 5 | | | | |
| Example 1 | 82.39 | 85.91 | 149 | −1.97 |
| Example 2 | 82.13 | 85.78 | 148 | −1.93 |
| Example 3 | 82.07 | 85.66 | 151 | −1.93 |
| Example 4 | 82.09 | 85.76 | 150 | −1.92 |
| Example 5 | 83.94 | 86.05 | 147 | −1.91 |
| Example 6 | 83.46 | 86.09 | 147 | −1.93 |
| Example 7 | 82.97 | 85.94 | 149 | −1.96 |
| Example 8 | 85.64 | 87.17 | 143 | −1.70 |

### SUMMARY

Referring to Table 2, if (e.g., when) either one type or kind of additive out of the two types (kinds) of additives was utilized, the thermal exposure was improved to 142° C., but if (e.g., when) the two types (kinds) of additives were combined, the thermal exposure was improved to 143° C., (e.g., by additionally 1° C. or more).

In contrast, referring to Table 3, if (e.g., when) the two types (kinds) of additives were combined, a content (e.g., amount) of each additive and a total content (e.g., amount) of the two additives should be limited by comprehensively considering storage characteristics at a relatively high temperature.

For example, based on a total amount of the electrolyte, the first additive should be utilized within a range of 5 to 20 wt %, the second additive should be utilized within a range of 0.1 to 2 wt %, and combined (e.g., all) the additives should be utilized within a range of 7.1 to 21 wt % in total.

A battery management system (BMS) device, and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, and/or the like. Also, a person of skill in the art should recognize that the functionality of various computing devices may be combined or integrated into a single computing device, or the functionality of a particular computing device may be distributed across one or more other computing devices without departing from the scope of the present disclosure.

While this invention has been described in connection with what is presently considered to be practical example embodiments, it is to be understood that present disclosure is not limited to the disclosed embodiments, but, on the contrary, is intended to cover one or more suitable modifications and equivalent arrangements included within the spirit and scope of the appended claims, and equivalents thereof.

### REFERENCE NUMERALS

100: rechargeable lithium battery
10: positive electrode
20: negative electrode
30: separator
40: electrode assembly
50: case
60: sealing member

What is claimed is:

1. An electrolyte comprising:
a non-aqueous organic solvent;
a lithium salt;
a first additive represented by Chemical Formula 1; and
a second additive represented by Chemical Formula 2: 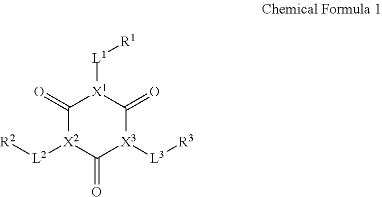
in Chemical Formula 1,
X¹ to X³ are each N or CH and at least one selected from among X¹ to X³ is N;
L¹ to L³ are each independently a single bond, or a substituted or unsubstituted C1 to C10 alkylene group; and
R¹ to R³ are each independently an epoxy group, or a substituted or unsubstituted C1 to C10 alkyl group, and at least one selected from among R¹ to R³ is an epoxy group; 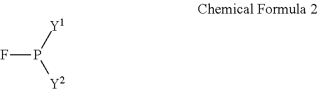
in Chemical Formula 2,
Y¹ and Y² are each independently a halogen or —O-L⁴-R⁴, and at least one selected from among Y¹ and Y² is —O-L⁴-R⁴;
L⁴ is a single bond or a substituted or unsubstituted C1 to C10 alkylene group;
R⁴ is a cyano group (—CN), a difluorophosphite group (—OPF₂), a substituted or unsubstituted C1 to C10 alkyl group, a substituted or unsubstituted C2 to C10 alkenyl group, a substituted or unsubstituted C3 to C10 cycloalkyl group, a substituted or unsubstituted C3 to C10 cycloalkenyl group, a substituted or unsubstituted C2 to C10 alkynyl group, a substituted or unsubstituted C3 to C10 cycloalkynyl group, or a substituted or unsubstituted C6 to C20 aryl group; and
when Y¹ and Y² are simultaneously —O-L⁴-R⁴, two R⁴s are not linked or are linked to form a substituted or unsubstituted monocyclic or polycyclic aliphatic heterocycle, or a substituted or unsubstituted monocyclic or polycyclic aromatic heterocycle,
wherein the electrolyte is for a rechargeable lithium battery.

2. The electrolyte as claimed in claim 1, wherein
X¹ to X³ are each N.

3. The electrolyte as claimed in claim 1, wherein
L¹ to L³ are each independently a substituted or unsubstituted C1 to C5 alkylene group.

4. The electrolyte as claimed in claim 1, wherein
R¹ to R³ are each an epoxy group.

5. The electrolyte as claimed in claim 1, wherein
the first additive is represented by Chemical Formula 1-1: 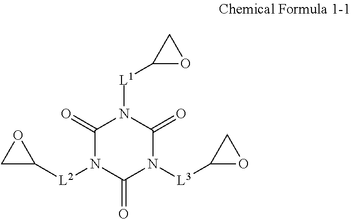
in Chemical Formula 1-1,
L¹ to L³ are each independently a substituted or unsubstituted C1 to C5 alkylene group.

6. The electrolyte as claimed in claim 1, wherein
the first additive is selected from among Chemical Formula 1-1-1 and Chemical Formula 1-1-2:

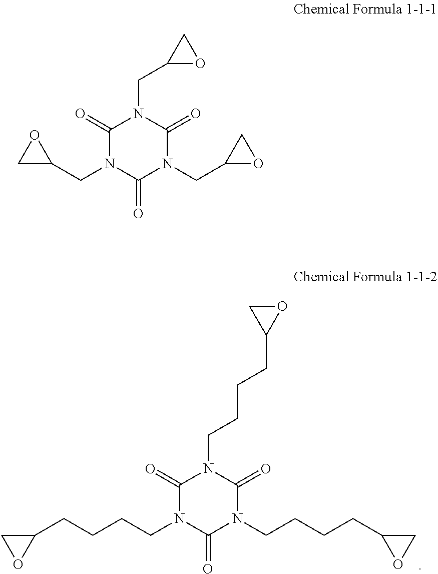 [SMILES: O=c1n(CC2CO2)c(=O)n(CC2CO2)c(=O)n1CC1CO1 ; O=c1n(CCCCC2CO2)c(=O)n(CCCCC2CO2)c(=O)n1CCCCC1CO1]

7. The electrolyte as claimed in claim 1, wherein:
one selected from among Y¹ and Y² is a fluoro atom and one selected from among Y¹ or Y² is —O-L⁵-R⁵, L⁵ is a single bond or a substituted or unsubstituted C1 to C10 alkylene group, and R⁵ is a cyano group (—CN) or a difluorophosphite group (—OPF₂); or
Y¹ is —O-L⁶-R⁶ and Y² is O-L⁷-R⁷, L⁶ and L⁷ are each independently a single bond or a substituted or unsubstituted C1 to C10 alkylene group, R⁶ and R⁷ are each independently a substituted or unsubstituted C1 to C10 alkyl group, and R⁶ and R⁷ are linked to form a substituted or unsubstituted monocyclic or polycyclic aliphatic heterocycle.

8. The electrolyte as claimed in claim 7, wherein
the second additive is represented by Chemical Formula 2-1 or Chemical Formula 2-2: 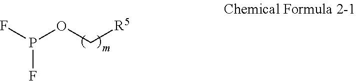
in Chemical Formula 1-1,
m is an integer of 1 to 5; and
R⁵ is —CN or —OPF₂, 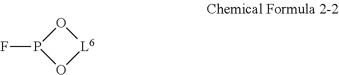
in Chemical Formula 1-2,
L⁶ is a substituted or unsubstituted C2 to C5 alkylene group.

9. The electrolyte as claimed in claim 8, wherein
the second additive is represented by Chemical Formula 2-2a or Chemical Formula 2-2b: 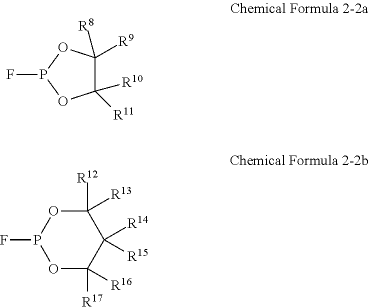
in Chemical Formulas 2-2a and 2-2b,
R⁸ to R¹⁷ are each independently a hydrogen atom, a halogen atom, or a substituted or unsubstituted C1 to C5 alkyl group.

10. The electrolyte as claimed in claim 1, wherein
the second additive is one selected from among:

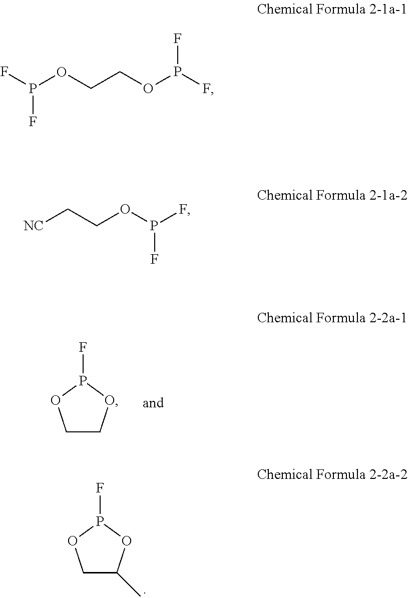

11. The electrolyte as claimed in claim 1, wherein
a weight ratio of the first additive to the second additive is about 2:1 to about 100:1.

12. The electrolyte as claimed in claim 1, wherein
the electrolyte comprises the first additive in an amount of about 5 to about 20 wt %, based on a total amount of the electrolyte.

13. The electrolyte as claimed in claim 1, wherein
the electrolyte comprises the second additive in an amount of about 0.1 to about 2 wt %, based on a total amount of the electrolyte.

14. The electrolyte as claimed in claim 1, wherein
the non-aqueous organic solvent comprises ethylene carbonate (EC):ethylmethyl carbonate (EMC):dimethyl carbonate (DMC).

15. A rechargeable lithium battery comprising:
a positive electrode comprising a positive electrode active material;
a negative electrode comprising a negative electrode active material; and
the electrolyte as claimed in claim 1.

16. The rechargeable lithium battery as claimed in claim 15, wherein
the rechargeable lithium battery further comprises a separator between the positive electrode and the negative electrode and impregnated with the electrolyte.

17. The rechargeable lithium battery as claimed in claim 15, wherein
the first additive is represented by Chemical Formula 1-1: 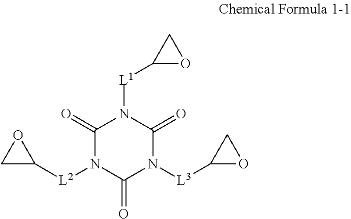
in Chemical Formula 1-1,
L¹ to L³ are each independently a substituted or unsubstituted C1 to C5 alkylene group.

18. The rechargeable lithium battery as claimed in claim 15, wherein:
one selected from among Y¹ and Y² is a fluoro atom and one selected from among Y¹ or Y² is —O-L⁵-R⁵, L⁵ is a single bond or a substituted or unsubstituted C1 to C10 alkylene group, and R⁵ is a cyano group (—CN) or a difluorophosphite group (—OPF₂); or
Y¹ is —O-L⁶-R⁶ and Y² is O-L⁷-R⁷, L⁶ and L⁷ are each independently a single bond or a substituted or unsubstituted C1 to C10 alkylene group, R⁶ and R⁷ are each independently a substituted or unsubstituted C1 to C10 alkyl group, and R⁶ and R⁷ are linked to form a substituted or unsubstituted monocyclic or polycyclic aliphatic heterocycle.

19. The rechargeable lithium battery as claimed in claim 15, wherein
the second additive is represented by Chemical Formula 2-1 or Chemical Formula 2-2: 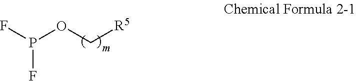
in Chemical Formula 1-1,
m is an integer of 1 to 5; and
R⁵ is —CN or —OPF₂, 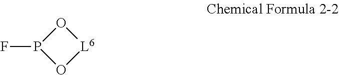
in Chemical Formula 1-2,
L⁶ is a substituted or unsubstituted C2 to C5 alkylene group.

20. The rechargeable lithium battery as claimed in claim 15, wherein
a weight ratio of the first additive to the second additive is about 2:1 to about 100:1.
